(19) United States
(12) Patent Application Publication
(10) Pub. No.: US 2026/0026715 A1
(43) Pub. Date: Jan. 29, 2026

(54) MULTI-CHANNEL AND MULTI-MODE AUDIOMETER AND METHOD

(71) Applicant: Mayo Foundation for Medical Education and Research, Rochester, MN (US)

(72) Inventors: Michael J. Cevette, Cave Creek, AZ (US); Gaurav N Pradhan, Fountain Hills, AZ (US)

(21) Appl. No.: 18/996,485

(22) PCT Filed: Jul. 20, 2023
(86) PCT No.: PCT/US2023/028227

§ 371 Date: Jan. 17, 2025

Related U.S. Application Data
(60) Provisional application No. 63/391,130, filed on Jul. 21, 2022.

(51) Int. Cl. A61B 5/12; A61B 5/00

(52) U.S. Cl. CPC A61B 5/123; A61B 5/128; A61B 5/445

(57) ABSTRACT

Enhanced audiometry methods and instruments to provide assessments of the nature and/or extent of a subject's hearing loss or impairments. Embodiments include applying at least first and second different test electrical stimulations to a subject, wherein the first and second test electrical stimulations are configured to stimulate hearing perception in the subject and are different. An identification of one or more characteristics of the perceived hearing produced by the at least first and second test electrical stimulations, such as an origin location of the perceived hearing, is received from the subject, and recorded in association with the at least first and second test electrical stimulations. Clinicians may improve the efficacy of treatments for the hearing impairments based on the recorded information.

### CROSS-REFERENCE TO RELATED APPLICATION

This application is a national phase application of PCT Application No. PCT/US2023/028227, internationally filed on Jul. 20, 2023, which claims the benefit of U.S. Application No. 63/391,130, filed Jul. 21, 2022, which are herein incorporated by reference in their entireties for all purposes.

### FIELD

This disclosure relates generally to devices and methods for diagnosing hearing or sound perception in subjects. Embodiments include audiometers and audiometry methods.

### BACKGROUND

Audiometry and instruments such as audiometers are used to assess hearing and sound perception in subjects. The results of audiometry may be used by clinicians to assess and treat hearing impairments.

There remains a continuing need for improved audiometry methods and instruments. In particular, there is a need for such methods and instruments that can provide enhanced hearing assessments, such as for example more extensive and/or detailed information on hearing and/or sound perception by subjects. Information provided by such enhanced assessments may, for example, enhance a clinician's ability to assess the nature and/or extent of a subject's hearing impairment, leading to enhanced treatments.

### SUMMARY

Enhanced audiometry methods and instruments are described in this disclosure. The methods and instruments provide enhanced assessments of the nature and/or extent of a subject's hearing loss or impairments. Using the enhanced assessments, clinicians may be able to improve the efficacy of treatments for the hearing impairments.

For example, by use of the audiometry methods and instruments, a clinician may evaluate the auditory system of a subject to determine a level of hearing loss and/or the site of lesion of a disorder. The technology can be used to evaluate the function of the auditory system under various applications. Applications include but are not limited to electrical speech and music perception using ear protection, noise cancellation effectiveness under earphones, frequency filtered speech for improved speech perception, comparisons of electrical and acoustic stimulation thresholds to determine a site of lesion with improved range of testing over conventional bone conduction, evaluation of the suppression of tinnitus through electrical stimulation, ototoxicity monitoring of high frequencies to for example 20 KHz, lateralization for tones and speech for asymmetrical hearing loss, the value of combined acoustic and electrical sound for speech enhancement, and portable hearing aid functional gain testing for cochlear hearing loss. The capability of altering timing, and levels of stimuli across multiple channels is provided to enable enhanced assessments. Determining delivery of sound at various locations can be provided for assessments. Enhanced central auditory testing and evaluation of temporal integration, and masking by manipulating time/intensity interplay, are also capable.

A first example in accordance with embodiments is an audiometry method. Embodiments may comprise: applying first test electrical stimulation to a subject, wherein the first test electrical stimulation is configured to stimulate hearing perception in the subject; applying second test electrical stimulation to the subject, wherein the second test electrical stimulation is configured to stimulate hearing perception in the subject and is different than the first test electrical stimulation; receiving from the subject an identification of one or more characteristics of the perceived hearing produced by the first test electrical stimulation and the second test electrical stimulation, wherein the one or more characteristics includes an origin location of the perceived hearing; and recording the one or more characteristics of the perceived hearing identified by the subject in association with the first test electrical stimulation and the second test electrical stimulation.

In some embodiments of the first example, applying the first test electrical stimulation and the second test electrical stimulation includes simultaneously applying the first test electrical stimulation and the second test electrical stimulation.

In any or all embodiments of the first example, applying the first test electrical stimulation and the second test electrical stimulation includes sequentially applying the first test electrical stimulation and the second test electrical stimulation.

In any or all embodiments of the first example, applying the first test electrical stimulation and/or the second test electrical stimulation includes applying electrical stimulation corresponding to one or more of (1) a tone, (2) a range of tones, (3) speech, or (4) background noise.

In any or all embodiments of the first example, applying the first test electrical stimulation and/or the second test electrical stimulation includes applying the electrical stimulation corresponding to a range of intensities; and receiving from the subject an identification of characteristics of the perceived hearing includes an identification of a threshold intensity at which the subject perceives the hearing.

In any or all embodiments of the first example, applying the first test electrical stimulation includes applying stimulation configured to at least predominately stimulate hearing on a first lateral side of the subject; and applying the second test electrical stimulation includes applying stimulation configured to at least predominately stimulate hearing on a second lateral side of the subject that is different than the first lateral side.

In any or all embodiments of the first example, applying the first test electrical stimulation includes applying the first electrical stimulation to a first lateral side mastoid region of the subject; and applying the second test electrical stimulation includes applying the second electrical stimulation to a second lateral side mastoid region of the subject.

In any or all embodiments of the first example, applying test electrical stimulation comprises applying electrical stimulation corresponding to a source of perceived hearing on a first lateral side of the subject.

In any or all embodiments of the first example, applying test electrical stimulation comprises applying electrical stimulation corresponding to a source of perceived hearing on a second lateral side of the subject.

In any or all embodiments of the first example, applying test electrical stimulation comprises applying electrical stimulation corresponding to a source of perceived hearing on an anterior side of the subject.

In any or all embodiments of the first example, applying test electrical stimulation comprises applying electrical stimulation corresponding to a source of perceived hearing on a posterior side of the subject.

In any or all embodiments of the first example, applying test electrical stimulation comprises applying electrical stimulation corresponding to a source of perceived hearing above the subject.

In any or all embodiments of the first example, applying test electrical stimulation comprises applying electrical stimulation corresponding to a source of perceived hearing below the subject.

In any or all embodiments of the first example, applying test electrical stimulation comprises applying electrical stimulation corresponding to a stationary source of perceived hearing.

In any or all embodiments of the first example, applying test electrical stimulation comprises applying electrical stimulation corresponding to a moving source of perceived hearing.

In any or all embodiments of the first example, the method further comprises: applying at least a third test electrical stimulation to the subject, wherein the third test electrical stimulation is different than the first and second test electrical stimulation; receiving from the subject an identification of characteristics of perceived hearing produced by the first, second and third test electrical stimulation; and recording the characteristics of the perceived hearing identified by the subject in association with the first, second and third test electrical stimulation.

In any or all embodiments of the first example, applying the first and second test electrical stimulation comprises applying first and second stimulation having alterations in timing between the first and second stimulation. For example, timing differences between multiple electrodes influence lateralization of sound and provide a complex auditory stimulation for the evaluation of the central auditory pathways.

In any or all embodiments of the first example, applying the first and second test electrical stimulation comprises applying first and/or second test electrical stimulation corresponding to increasing intensity with changing frequency.

A second example in accordance with embodiments is an audiometry method in accordance with any or all of the embodiments of the first example, further comprising: applying one or more of: test bone conduction stimulation to the subject, wherein the test bone conduction stimulation is configured to stimulate hearing perception in the subject; or test air-conducted stimulation to the subject, wherein the test air-conducted stimulation is configured to stimulate hearing perception in the subject; receiving from the subject an identification of characteristics of perceived hearing produced by the first and second test electrical stimulation and the one or more of the test bone conduction stimulation and the test air-conducted stimulation; and recording the characteristics of the perceived hearing identified by the subject in association with the first and second test electrical stimulation and the one or more of the test bone conduction stimulation or the test air-conducted stimulation.

A third example in accordance with embodiments is an audiometry method in accordance with any or all of the embodiments of the first and second examples, wherein applying the first and second test electrical stimulation includes: applying the stimulation to at least predominately stimulate hearing on a first lateral side of the subject; and applying the stimulation to mask the hearing on a second lateral side of the subject that is different that the first lateral side and that is produced by the stimulation on the first lateral side of the subject.

Any or all embodiments of the third example may further comprise attenuating air conducted hearing of the subject while performing the method.

In any embodiments of the third example, attenuating air conducted hearing includes applying earplugs, ear muffs or other physical sound attenuating structures to one or more ears of the subject.

In any embodiments of the third example, attenuating air conducted hearing includes applying sound cancelling air conducted stimulation to one or more ears of the subject.

A fourth example in accordance with embodiments is a method in accordance with any or all embodiments of the first, second and/or third examples, wherein the method is performed while the subject's ears are protected from ambient sound, for example by earmuffs, earplugs or noise cancellation.

In embodiments of the fourth example, the method is used for diagnosing tinnitus.

In embodiments of the fourth example, the method is used for diagnosing a site of lesion in the subject's ear organ, for example the outer ear, middle ear, or inner ear.

### BRIEF DESCRIPTION OF THE DRAWINGS

FIG. 1 is a diagrammatic representation of an audiometry system in accordance with embodiments.

FIGS. 2A and 2B are diagrammatic illustrations of headsets including multi-channel and multi-mode stimulation devices located with respect to a head of a subject, in accordance with embodiments.

FIG. 3 is a diagrammatic illustration of functional components of a multi-channel and multi-mode audiometer, in accordance with embodiments.

FIG. 4 is a diagrammatic illustration of components of a computer system that may be used to implement the audiometer shown in FIG. 3, in accordance with embodiments.

### DETAILED DESCRIPTION

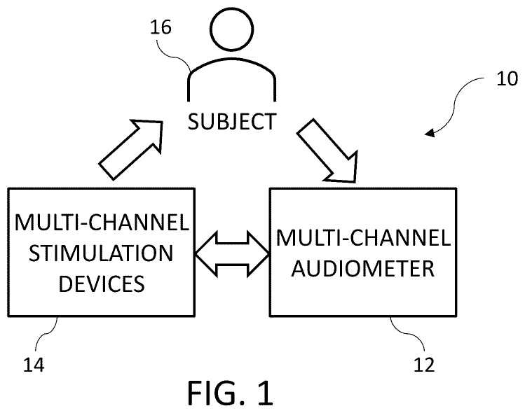

FIG. 1 is a diagrammatic illustration of an audiometry system 10 in accordance with embodiments. As shown, system 10 includes a multi-channel and multi-mode audiometer 12, and multi-channel and multi-mode stimulation devices 14. Audiometer 12, which may be operated by a clinician, generates multi-channel and optionally multi-mode stimulation signals that are coupled to the stimulation devices 14. The stimulation signals cause the stimulation devices 14 to produce multi-channel and optionally multi-mode stimulation that is applied to a subject 16 for purposes of stimulating and causing hearing and/or the perception of hearing by the subject. The subject 16 may describe hearing or sound information representative of characteristics, such as for example the nature, amplitude or level, location, and/or origin or direction of source of the sound or perceived hearing produced by the stimulation. The sound information provided by the subject 16 may be recorded, for example to or by the audiometer 12, typically in association with the corresponding stimulation. In embodiments, the multi-channel stimulation includes first and second channel electrical stimulation (e.g., electrical mode stimulation), where the first and second electrical stimulation are different. Embodiments optionally include one or more channels of one or more additional modes of stimulation, such as bone conduction stimulation and/or air-conducted or audio stimulation. The recorded sound information provided by the subject 16 in response to the multi-channel, and any multi-mode stimulation, may be used by a clinician to provide efficacious assessments of any hearing impairments of the subject 16. The recorded sound information may also be used to determine effective approaches for providing sound or hearing perception to the subject 16, for example in situations where the subject is unable to perceive air conducted hearing via their ear organs.

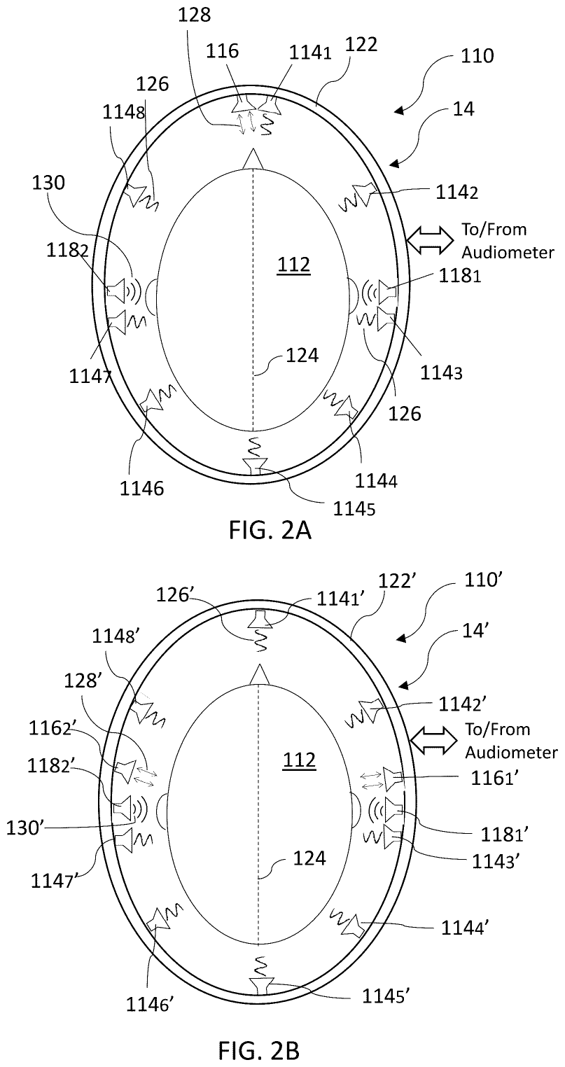

FIG. 2A is a diagrammatic illustration of a structure such as headset 110 including multi-channel stimulation devices 14, and that can be used to position the stimulation devices 14 with respect to a head 112 of the subject 16, in accordance with embodiments. The illustrated embodiments of headset 110 are configured for multi-channel and multi-mode stimulation, and include a plurality (eight are shown for purposes of example) of electrodes 1141-1148 for electrical stimulation, one bone conduction transducer 116 for bone conduction stimulation, and a plurality (two are shown for purposes of example) of air conducted, pressure wave or audio transducers 1181-1182 for air-conducted stimulation, all of which are mounted to a band 122 configured to be attached to or positioned with respect to the head 112 of the subject 16. Lateralization, or the location or direction of the perceived sound caused by the stimuli produced by the electrodes 1141-1148, bone conduction transducer 116 and/or audio transducers 1181-1182. may depend on factors such as the numbers and/or locations of the electrodes, bone conduction transducers or audio transducers. Accordingly, other embodiments include electrodes such as 1141-1148, bone conduction transducers such as 116 or audio transducers such as 1181-1182 in other combinations of numbers and/or locations. For example, embodiments may include only two or only four electrodes such as 1141-1148, and in such embodiments the two or four electrodes may be located near the cochlea of the subject's head 112 (e.g., at the mastoids).

In the embodiments shown in FIG. 2A, for example, electrodes 1141-1148 are positioned on the band 122 at locations such that they will be located at adjacent positions corresponding to 12:00, 1:30, 3:00, 4:30, 6:00, 7:30, 9:00 and 10:30, respectively, on the head 112 of the subject. Electrodes 1143 and 1147 may be located at positions adjacent the right and left mastoids of the head 112 of the subject 16 to optimize or otherwise enhance the ability of electrical stimulation provided by the electrodes to stimulate the right and left cochlea, respectively, of the subject. Electrodes 1141 and 1145 are shown located at positions that will be adjacent the anterior and posterior portions of the subject's head 112 (e.g., at the ends of a medial midline 124 through the subject's head). As described in greater detail below, electrodes 1141-1148 produce electrical signals (shown diagrammatically at 126 in FIG. 2A) that stimulate the cochlea of the subject 16 to cause the perception of hearing or sound. Electrodes 1141-1148 may be conventional or otherwise known devices suitable for the functions and applications described herein. Nonlimiting examples include surface electrodes typically used for stimulation and recording of electrical signals. Although described as devices that provide transcutaneous electrical stimulation in this disclosure, it will be appreciated that electrodes such as 1141-1148 may be utilized in devices in other embodiments.

Bone conduction transducer 116 is shown located at a position that will be adjacent the anterior portion of the subject's head 112 (e.g., on the subject's forehead at the medial midline 124). As described in greater detail below, bone conduction transducer 116 produces physical movement or other vibratory actions (shown diagrammatically at 128 in FIG. 2A) that vibrate the skull and/or other anatomical structures of the subject's head 112 to stimulate and cause the perception of hearing or sound. Bone conduction transducer 116 may include conventional or otherwise known devices suitable for the functions and applications described herein. Nonlimiting examples include devices producing mechanical oscillation or vibration.

Audio transducers 1181-1182 produce audible and air conducted pressure waves (shown diagrammatically at 130 in FIG. 2A) that vibrate the eardrum, tympanic membrane and/or other anatomical structures of the ear or auditory system in the subject's head 112 to produce hearing or the perception of hearing or sound. The audio transducers 1181-1182 may be located at positions adjacent the ear canals of the subject to optimize the hearing they produce. Audio transducers 1181-1182 may be conventional or otherwise known devices suitable for the functions and applications described herein. Nonlimiting examples include audio speakers, such as for example of the types used in hearing aids.

FIG. 2B is a diagrammatic illustration of exemplary alternative embodiments of a headset 110′. As shown, headset 110′ includes a plurality of electrodes 1141′-1148′, two bone conduction transducers 1161′ and 1162′, and a plurality of audio transducers 1181′-1182′, all of which are mounted to a band 122′ configured to be attached to or positioned with respect to the head 112 of the subject 16. In the embodiments shown in FIG. 2B, the two bone conduction transducers 1161′ and 1162′ are shown located at positions on the band 122′ such that they will be located adjacent positions corresponding to 3:00 and 2:00, respectively on the head 112 of the subject 16. Other than these different numbers and locations of the bone conduction transducers 1161′ and 1162′, headset 110′, including the electrodes 1141′-1148′ and audio transducers 1181′-1182′, may be the same as or similar to, and located at or adjacent positions that are the same as or similar to, those described in connection with FIG. 2A. Similar reference numbers are used to indicate similar features in FIGS. 2A and 2B. Throughout this disclosure the terms “stimulation devices” or “stimulation transducers” may be used to refer to one or more, or all, of stimulation devices, such as for example one or more the stimulation devices 1141-1148, 1141′-1148′, 116, 1161′, 1162′, 1181-1182, or 1181′-1182′.

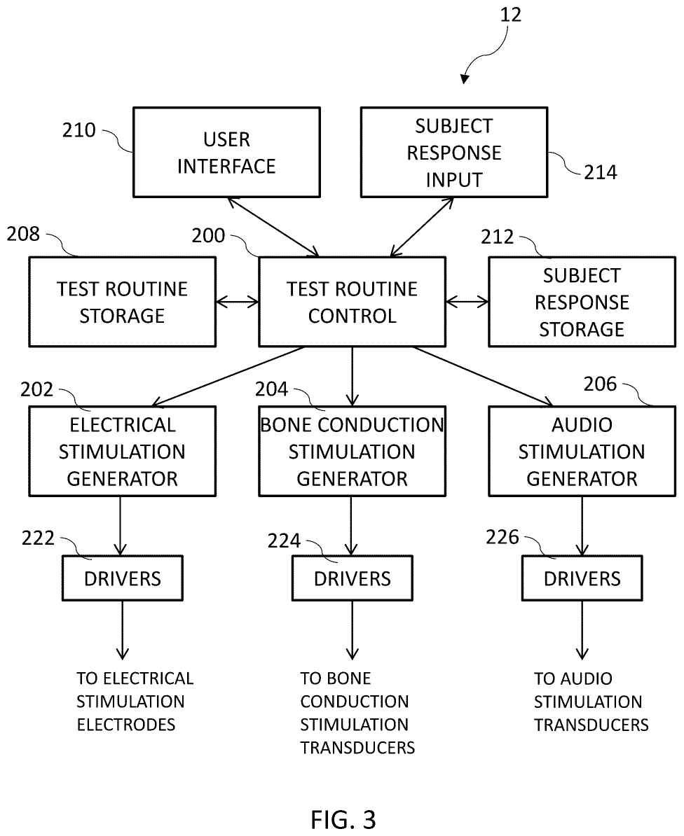

FIG. 3 is a diagrammatic illustration of functional components of an audiometer 12 that can be used in connection with stimulation devices 14 such as those described above in connection with FIGS. 2A and 2B to provide multi-channel and optionally multi-mode diagnostic hearing perception stimulation in accordance with embodiments. The illustrated embodiments of audiometer 12 include test routine control 200 coupled to electrical stimulation generator 202, bone conduction stimulation generator 204 and air conducted or audio stimulation generator 206. Information characterizing audiometry diagnostic routines or tests that can be performed by the audiometer 12 may be stored in test routine storage 208 that is coupled to the test routine control 200. User interface 210 that is coupled to the test routine control 200 may be used by a clinician or other user to control the operation of the audiometer 12. Nonlimiting examples of physical and/or functional components of the user interface 210 include knobs, buttons, switches or graphical user interface (GUI) components, including displays, such as for example to select and control functions, audiometry test routines and parameters and associated information, to control levels, and to provide information (e.g., visually) to a clinician or other operator or user of the audiometer 12. Information included in responses from the subjects to the audiometry test routines provide by the audiometer 12 may be stored in subject response storage 212. The embodiments illustrated in FIG. 3 include a subject response input 214 through which information in the subject responses to the test routines can be received by the audiometer 12. In embodiments, for example, the subject response input 214 may include a user input including components such as a keypad and display that can be operated by the subject or clinician to enter the subject responses. Alternatively or additionally, subject response input 214 may be a communication interface (e.g., a wired or wireless interface) couplable to another device (not shown) operated by the subject or clinician to receive information representative of the subject responses provided via that other device. Alternatively or additionally, the user interface 210 may be used by a clinician to enter the subject responses and other information.

Audiometer 12 supports a number of different diagnostic test modes, including one or more of an electrical stimulation test mode, a bone stimulation test mode, and an air conduction test mode. As described in greater detail below, an efficacious feature of embodiments of audiometer 12 is its capability of supporting multi-mode diagnostic test modes such as for example an electrical and bone stimulation test mode, an electrical and air conduction stimulation test mode, and an electrical, bone and air conduction stimulation test mode. An operator can, for example, use the user interface 210 to select a desired test mode.

Audiometry test routines stored by the test routine storage 208 include electrical stimulation routines, bone stimulation routines and air conduction stimulation routines. Depending on the selected test mode, the audiometer 12 will operate in accordance with one or more of the test routines stored by the test routine storage 208 and optionally in response to control parameters provided by a clinician (e.g., through the user interface 210). In embodiments, an operator can use the user interface 210 to select the desired test routines to be performed during the selected test mode. Alternatively or additionally, audiometer 12 may be configured with predetermined test routines for one or more of the test modes. During the multi-mode test modes, two or more of the electrical stimulation routines, bone stimulation routines or air conduction stimulation routines may be performed simultaneously and/or sequentially by the audiometer 12.

Electrical stimulation generator 202 generates multi-channel electrical stimulation signals based on the multi-channel electrical stimulation routines stored in test routine storage 208 and control parameters provided by the operator. The multi-channel electrical stimulation signals are configured to be coupled to two or more of the electrodes such as 1141-1148 or 1141′-1148′of the stimulation devices 14. Electrical stimulation generator 202 produces the electrical stimulation signals in forms and having characteristics configured to cause the electrodes to electrically stimulate the cochlea in the head 112 of the subject 16, and thereby the perception of sound or hearing in the subject.

The multi-channel stimulation provided by the electrical stimulation generator 202 may provide lateralization (e.g., directional) hearing perception in the subject 16. In embodiments, electrical stimulation generator 202 may generate multi-channel electrical stimulation signals that are applied to transducers such as 1141-1148, in such a manner that the sound perceived by the subject 16 is perceived to originate from one or more locations or directions, and at levels, corresponding to locations or directions of a sphere surrounding the head 112 of the subject 16. For example, the multi-channel electrical stimulation signals may have forms and characteristics (e.g., levels and phases), and are applied to specific electrodes, that cause the hearing to be perceived as originating from specific locations or directions around the head 112 of the subject 16 (e.g., at 12:00, 3:00, 6:00 and/or 9:00 positions, above, below, or other locations or directions with respect to a sphere about the head of the subject). Electrical stimulation generator 202 can be configured to produce the electrical simulation signals by conventional or otherwise known approaches. Electrical stimulation approaches of these types are also sometimes referred to as galvanic vestibular stimulation (GVS) or cochlear stimulation. For example, the Puharich U.S. Pat. No. 3,267,931, Zink U.S. Pat. No. 3,766,331 and Tonndorf et al. article entitled High Frequency Audiometry published in 1984 describe approaches for modulating carrier signals to produce electrical stimulation signals that, when applied to the cochlea through electrodes, will produce electrical signals causing stimulation of hearing perception. The Puharich and Zink patents and the Tonndorf et al. article are hereby incorporated by reference and for all purposes. Electrical stimulation modalities of these types are generally capable of stimulating perceived sound over a relatively wide range of frequencies corresponding to much or all of the full range of typical hearing, such as 100 Hz to 20,000 Hz.

Embodiments of bone conduction stimulation generator 204 generate mono-channel or single-channel bone conduction stimulation signals based on the bone stimulation routines stored in the test routine storage 208 and control parameters provided by the operator. When used in connection with a headset such as 110, for example, the mono-channel bone conduction stimulation signals are configured to be coupled to the bone conduction transducer 116. When used in connection with a headset such as 110′, for example, the mono-channel bone conduction stimulation signals are configured to be coupled to one or both of the bone conduction transducers 1161′ and 1162′. Bone conduction stimulation generator 204 produces the bone conduction stimulation signals in forms and having characteristics configured to cause the bone conduction transducers to vibrate the skull of the head 112 of the subject 16, and thereby cause the perception of hearing in the subject. Bone conduction stimulation of the subject 16 provided by bone conduction stimulation signals produced by the bone conduction stimulation generator 204, when provided concurrently with (e.g., simultaneously and/or sequentially or otherwise in combination with) one or both of the multi-channel electrical stimulation produced through the electrical stimulation generator 202 or the audio stimulation produced through the audio stimulation generator 206, provides the capability of evaluating and diagnosing relatively detailed and complex components of the subject's ability to perceive sound, including lateralization.

Embodiments of bone conduction stimulation generator 204 generate multi-channel bone conduction stimulation signals based on the bone stimulation routines stored in the test routine storage 208 and control parameters provided by the operator. When used in connection with a headset such as 110′, for example, the multi-channel bone conduction stimulation signals are configured to be coupled to the bone conduction transducers 1161′ and 1162′. Bone conduction stimulation generator 204 produces the bone conduction stimulation signals in forms and having characteristics configured to cause the bone conduction transducers to vibrate the skull of the head 112 of the subject 16, and thereby cause the perception of hearing in the subject.

The multi-channel stimulation provided by the bone conduction stimulation generator 204 may provide lateralization hearing perception in the subject. In embodiments, bone conduction stimulation generator 204 may generate multi-channel bone conduction stimulation signals that are applied to transducers such as 1161′ and 1162′, in such a manner that the sound perceived by the subject 16 is perceived to originate from one or more locations or directions, and at levels, corresponding to locations or directions of a sphere surrounding the head 112 of the subject 16. For example, the multi-channel bone conduction stimulation signals have forms and characteristics, and are applied to specific transducers, that cause the hearing to be perceived as originating from specific locations or directions around the head 112 of the subject 16 (e.g., at 12:00, 3:00, 6:00 and/or 9:00 positions, above, below, or other locations or directions with respect to a sphere about the head of the subject). In at least some instances it has been observed that lateralization (e.g., directional and level) capabilities provided by the multi-channel bone conduction stimulation may be less than the capabilities provided by electrical stimulation or audio stimulation modalities. However, multi-channel bone conduction stimulation of the subject 16 provided by bone conduction stimulation signals produced by the bone conduction stimulation generator 204, when provided concurrently or otherwise in combination with one or both of the multi-channel electrical stimulation produced through the electrical stimulation generator 202 or the audio stimulation produced through the audio stimulation generator 206, provides enhanced capabilities for evaluating and diagnosing relatively detailed and complex components of the subject's ability to perceive sound, including lateralization.

Bone conduction stimulation generator 204 can be configured to produce the bone conduction stimulation signals by conventional or otherwise known approaches. For example, the bone conduction stimulation signals may have varying frequencies corresponding to or representative of the desired sound perception to be provided to the subject. The bone conduction stimulation signals may be amplitude modulated or frequency modulated and/or phased in embodiments. The following articles, for example, describe various bone conduction stimulation approaches that may be used in connection with bone conduction stimulation generator 204, and are incorporated herein in their entireties and for all purposes: Shiraishi, K., Sound Localization and Lateralization by Bilateral Bone Conduction Devices, Middle Ear Implants and Cartilage Conduction Hearing Aids, Audiology Research 2021, 11, 508-523; Stanley, R. et al., Lateralization of Sounds Using Bone-Conducted Headsets, Proceedings of the Human Factors and Ergonomics Society 50^{th} Annual Meeting 2006; Daga, K. et al., Bone-Conducted Sound Lateralization of Interaural Time Difference and Interaural Intensity Difference in Children and a Young Adult with Bilateral Microtia and Atresia of the Ears, Acta Otolaryngol. 2001, 121, 274-277. Bone conduction stimulation modalities of these types are typically capable of providing perception of hearing over frequency ranges below the upper ranges of those provided by electrical and air-conducted stimulation modalities. For example, the most effective frequency ranges of bone conduction stimulation in certain subjects may typically range from about 250 Hz to about 6,000 Hz.

Embodiments of audio stimulation generator 206 generate mono-channel air conducted stimulation signals based on the audio stimulation routines stored in the test routine storage 208 and control parameters provided by the operator. In certain embodiments the audio stimulation generator 206 generates multi-channel audio stimulation signals based on audio stimulation routines stored in the test routine storage 208 and control parameters provided by the operator. When used in connection with headsets such as 110 or 110′, for example, the mono-channel or multi-channel audio stimulation signals are configured to be coupled to one or both of the audio transducers 1181-1182. or one or both of the audio transducers 1181′-1182′. Audio stimulation generator 206 produces the audio stimulation signals in forms and having characteristics configured to cause the audio transducers 1181-1182 or 1181′-1182′ to produce audible sound via air conducted pressure waves, and thereby hearing in the subject 16. Audio stimulation of the subject 16 provided by audio stimulation signals produced by the audio stimulation generator 206, when provided concurrently with or otherwise at the same time as (e.g., simultaneously and/or sequentially) one or both of the multi-channel electrical stimulation produced by the electrical stimulation generator 202 or mono-channel or multi-channel bone conduction stimulation produced by the bone conduction stimulation generator 204 provides enhanced capabilities of evaluating and diagnosing relatively detailed and complex components of the subject's ability to perceive sound, including lateralization.

Multi-channel stimulation provided by the audio stimulation generator 206 may provide lateralization hearing perception in the user. In embodiments, audio stimulation generator 206 may generate multi-channel audio stimulation signals that are applied to transducers such as 1181 and 1182, or 1181′ and 1182′, in such a manner that the sound perceived by the subject 16 is perceived to originate from one or more locations or directions, and at levels, corresponding to locations or directions of a sphere surrounding the head 112 of the subject 16. For example, the multi-channel audio stimulation signals may have forms and characteristics, and may be applied to specific audio transducers, that cause the hearing to be perceived as originating from specific locations or directions around the head 112 of the subject 16 (e.g., at 12:00, 3:00, 6:00 and/or 9:00 positions, above, below, or other locations or directions with respect to a sphere about the head of the subject).

Audio stimulation generator 206 can be configured to produce the audio stimulation signals by conventional or otherwise known approaches. For example, the audio stimulation signals may be frequency modulated and/or amplitude modulated and/or phased in a manner that corresponds to the frequencies and/or levels of the desired sound perception to be provided to the subject. Nonlimiting examples include approaches used by conventional or otherwise known hearing aids. Audio stimulation modalities of these types are typically capable of stimulating perceived sound over a relatively wide range of frequencies corresponding to much or all of the full range of typical hearing, such as 100 Hz to 20,000 Hz.

The embodiments of the audiometer 12 shown in FIG. 3 include drivers 222, 224 and 226 coupling the electrical stimulation generator 202, bone conduction stimulation generator 206 and audio stimulation generator 208, respectively, to the associated stimulation devices such as electrical stimulation electrodes 1141-1148 or 1141′-1148′, bone conduction stimulation transducers 116 or 1161′ or 1162′, or audio stimulation transducers 1181-1182 or 1181′-1182′. Drivers 222, 224 and/or 226 convert the signals produced by the respective signal generators 202, 204 or 206 to levels suitable for application to the associated stimulation devices. In embodiments, for example, drivers 222, 224 and 226 may include transformers or other components to convert the signals produced by the respective stimulation generators 202, 204 or 206 to current and/or voltage levels and/or impedance levels suitable for application to the stimulation devices.

The multi-channel electrical stimulation routines may include routines that produce electrical stimulation signals configured to provide a number of different types of predetermined or expected perceived hearing sensations in a subject 16 for purposes of diagnosing the subject's perceived hearing capabilities and limitations. In connection with these different types of electrical stimulation routines, the routines may produce electrical stimulation that when applied to the electrical stimulation electrodes produce current flow in a plurality of different directions through the head 112 of the subject 16. In general, increasing amplitudes of the current will correspond to increasing intensity (e.g., sound pressure level or SPL) of the stimulated hearing. These different current flow direction signals can be configured to produce sound perception from different locations with respect to the head 112 of the subject 16. For example, when using two electrodes such as 1143 and 1147 located adjacent the right and left mastoids, respectively, of the subject 16, stimulation signals applied in the right to left current flow direction (e.g., a first direction, from anode to cathode) produce sound perception in the right side of the subject, and stimulation signals applied in the left to right current flow direction (e.g., a second direction, from anode to cathode) produce sound perception in the left side of the subject. By this approach the two electrodes are used as both an anode and a cathode during different, for example sequential, time periods. In other embodiments these multiple current flow direction signals can be provided, for example simultaneously, by the use of four electrodes (e.g., one anode and one cathode at each of two locations). Stimulation routines that use other combinations of electrical stimulation electrodes and/or different (e.g., first and second) direction current flow electrical stimulation signals can be configured produce expected perception of hearing from any of a wide range of source directions or locations with respect to the head 112 of the subject 16. For example, the electrical stimulation signals may be configured to predominately stimulate the perception of hearing at one or more of a first or right lateral side, a second or left lateral side, an anterior side, a posterior side, above, or below with respect to the head 112 of the subject 16. The electrical stimulation routines may be configured to produce the perception of a static or stationary source of perceived hearing, and/or or the perception of a dynamic or moving source of perceived hearing.

Electrical stimulation routines may be configured to produce electrical stimulation signals that provide perceptions corresponding to different types or natures of sounds, such as for example one or more of a tone, a range of tones, speech, or background noise. The electrical stimulation signals may be configured to provide perceptions corresponding to a range of amplitudes or intensities. For example, the intensities may increase or decrease. As another example, the electrical stimulation routines may be configured to provide each of a plurality of different types of natures at sounds that originate at each of a plurality of different locations, including moving locations, over a range of amplitudes or intensities that encompasses levels below a threshold that the subject can perceive and levels above a threshold the subject can perceive. For example, the electrical stimulation signals can be configured to increasing or decreasing intensity with changes (e.g., increases or decreases) in frequency of the sound. Any changing or dynamic characteristics of the perceived sounds (e.g., source, sound type and/or intensity) can be set or predetermined by the stored data characterizing the electrical stimulation routines, or controllable by the operator, for example through use of the user interface 210.

Similarly, the bone conduction stimulation routines and air conducted stimulation routines may include routines that produce bone conduction stimulation signals and/or air conducted stimulation configured to provide one or more different types of predetermined or expected perceived hearing sensations in a subject 16 for purposes of diagnosing the subject's perceived hearing capabilities and limitations. In connection with these different types of bone conduction and air conducted stimulation routines, the routines may produce stimulation signals configured to be applied to the stimulation transducers at one or more locations on the head 112 of the subject 16. The bone conduction and air conducted stimulation may be configured to produce sound perception from different locations with respect to the head 112 of the subject 16. For example, bone conduction stimulation signals applied to the transducer 116 of the headset 110 may produce the perception of sound from locations along the medial midline 124 of the head 112 of the user 16 (e.g., in front of or above the head of the user). When using a headset such as 110′, bone conduction stimulation may be configured to produce sound perception from locations on different sides of the head 112 of the user 16 (e.g., on different sides of the medial midline 124) by controlling the characteristics such as the relative amplitudes or levels and phasing of the bone conduction stimulation signals applied to the transducers 1161′ and 1162′ on the opposite sides of the user's head. For example, the bone conduction stimulation signals may be configured to predominately stimulate the perception of hearing at one or more of a first or right lateral side, a second or left lateral side, or an anterior side with respect to the head 112 of the subject 16. Similarly, the air conducted stimulation signals applied to transducers 1181 and 1182 or 1181′ and 1182′ may be configured to produce sound perception from different locations with respect to the head 112 of the subject 16, such as for example on different sides of the head 112 of the user 16 (e.g., on different sides of the medial midline 124) by controlling the characteristics such as the relative amplitudes or levels and phasing of the air conduction stimulation signals applied to the transducers on the opposite sides of the user's head. For example, the air conduction stimulation signals may be configured to predominately stimulate the perception of hearing at one or more of a first or right lateral side, a second or left lateral side, or an anterior side with respect to the head 112 of the subject 16. The bone conduction and/or air conduction stimulation routines may be configured to produce the perception of a static or stationary source of perceived hearing, and/or or the perception of a dynamic or moving source of perceived hearing.

The bone conduction and air conduction stimulation routines may be configured to produce stimulation signals that may be expected to provide perceptions corresponding to different types or natures of sounds, such as for example one or more of a tone, a range of tones, speech, or background noise. The bone conduction and air conduction stimulation signals may be configured to provide expected perceptions corresponding to a range on amplitudes or intensities. For example, the intensities may increase or decrease. As another example, the bone conduction and/or air conduction stimulation routines may be configured to provide each of a plurality of different types of natures at sounds that originate at each of a plurality of different locations, including moving locations, over a range of amplitudes or intensities that encompasses levels below thresholds that the subject can perceive and levels above thresholds the subject can perceive. Any desired changing or dynamic characteristics of the perceived sounds (e.g., source, sound type and/or intensity) can be set or predetermined by the stored data characterizing the bone conduction or air conducted stimulation routines, or controllable by the operator, for example through use of the user interface 210.

In response to the use of audiometer 12 to stimulate perceived hearing in the subject 16 via the stimulus provided by the test routines of the test modes, the subject can provide information describing or characterizing the hearing or sound they perceive. The information provided by the subject 16 may, for example, characterize the nature of the perceived hearing (e.g., whether it is a tone, a range of tones, speech or background noise), the level or intensity of the perceived hearing, or locations of the source or origin of the perceived hearing (e.g., a left or right side, in front of, behind, above or below, and/or whether it is moving). For example, the subject may identify the point in time that the perceived hearing of a stimulation routine crosses a threshold intensity level from level that they are unable to perceive to a level that they are able to perceive. As described above, the perceived hearing information may, for example, be received from the subject directly (e.g., via subject response input 214) or indirectly (e.g., verbally from the subject to the operator of the audiometer 12, with the operator of the audiometer entering the responsive hearing information via the use interface 210). The hearing information provided by the subject 16 may be stored in the subject response storage 212. In embodiments, the subject response hearing information is stored in a manner that is associated with or registered to information describing corresponding to the characteristics (e.g., nature, intensity, location) of the stimulus that produced the hearing information.

Embodiments of audiometer 12 are configured to generate audiometry test reports or other information characterizing the hearing information stored in the subject response storage 212. The audiometry reports may include information describing the associated characteristics of the stimulus that resulted in the hearing information from the subject 16. For example, the audiometry reports may be presented to the clinician or other user through the user interface 210. Additionally or alternatively, the audiometry reports may be communicated (e.g., via wired or wireless communication interface) to a peripheral device such as a monitor, printer or mobile device, for review.

Audiometer 12, and audiometry test reports such as those described above, may be used by clinicians or other users to evaluate the auditory system of the user 16 and to determine the nature and/or degrees of any limitations in the user's auditory system. For example, the clinician may determine levels of hearing loss and/or the sites of lesion of a disorder (e.g., is hearing loss caused by structural or other conditions, disease or injury to the middle and/or inner ear). The diagnostics can be performed in accordance with any of a number of different applications. Such applications include, but are not limited to, electrical speech and music perceptions using ear protection, noise cancellation effectiveness when using earphones, frequency filtered speech for improved speech perception, comparisons of electrical and air conducted thresholds to determine sites of lesions. These and other diagnostics can be performed with improved ranges over conventional single mode and/or single channel diagnostic approaches. Suppression to tinnitus though electrical stimulation may be performed, as well as ototoxicity monitoring of relatively high frequencies to 20 KHz. Lateralization, for example for tones and speech, may be used to assess asymmetrical hearing loss. The value of multiple modes of stimulation, such as the combination of electrical stimulation with one or both or bone conduction stimulation or air conducted stimulation, can be assessed for purposes such as speech enhancement. Hearing aid functional gain testing for cochlear loss can be performed, for example in a portable manner. The capability of altering timing and levels of stimulation across multiple channels and/or modes enhances the types and efficacy of diagnoses that can be made. The perception of sound from a wide range of locations around the subject body can be assessed. Enhanced central auditory testing evaluating temporal integration and masking by manipulating time and intensity interplay can be performed.

For example, audiometer 12 may be used in applications to evaluate perceived hearing when air conducted hearing of the user is attenuated or masked, such as for example by ear protection (e.g., ear plugs or ear muffs) or in response to air conducted noise cancellation (e.g., as provided through headphones). While the air conducted hearing capabilities of the subject 16 are attenuated, the electrical stimulation and/or bone conduction stimulation may be applied to the subject. The electrical stimulation and/or bone conduction stimulation may, for example, have a speech or background noise nature, and the frequencies and/or intensities of the stimulation may be varied (e.g. independently). By the application of these test routines and through evaluation of the hearing response information obtained from the subject 16, stimulation modalities and characteristics that provide optimized perception of speech or other desired sound to the subject can be determined. Similarly, electrical stimulation and/or bone conduction stimulation test routines may be applied to a subject 16 while the subject is in a noisy environment (e.g., produced by air-conducted stimulation test routines representing a noisy environment). Hearing response information obtained from the subject 16 in response to the test routines may be used to determine stimulation modalities and characteristics that provide optimized perception of speech or other desired sound to the subject in air-conducted noisy environments. The determinations may be made in a range of application settings, such as quiet, noisy and in noise-cancelled settings. Applications of these types can be performed with the attenuation applied to one or both of the ears of the subject for lateralization-specific diagnoses or assessments, for example, as it pertains to evaluations of central auditory processing.

As other example, the responses of a subject 16 to various test routines may be used to determine stimulation modalities and characteristics that minimize or otherwise reduce tinnitus in a subject 16, for example, when the stimulus parameters such as level of current and frequency influence the perception of tinnitus.

As another example, electrical test routines can include multiple channel stimulation that provides alterations in timing of the stimulus between the channels, for example, in an effort to provide complex auditory stimulation for the evaluation of disorders of the central auditory system and the influence of stimulation on lateralization on binaural and single-sided deafness.

As another example, the sound may be delivered through speakers in a room rather than through earphones or electrodes attached to the head.

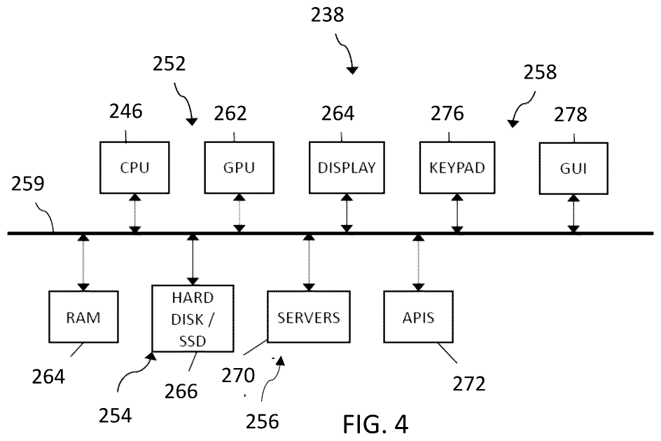

FIG. 4 is a diagrammatic illustration of an exemplary computer system 238 that may be used to implement the functional components of the audiometer 12. The illustrated embodiments of computer system 238 comprise processing components 252, storage components 254, network interface components 256 and user interface components 258 coupled by a system network or bus 259. Processing components 252 may, for example, include central processing unit (CPU) 260 and graphics processing unit (GPU) 262, and provide the processing functionality for the test routine control 200 and stimulation generators 202, 204 and 206. The storage components 254 may include RAM memory 264 and hard disk/SSD memory 266, and provide the storage functionality of the test routine storage 208 and the subject response storage 212. For example, operating system software used by the processing components 252 to implement methods described herein may be stored by the storage components 254. In embodiments, the network interface components may include one or more web servers 270 and one or more application programming interfaces (APIs) 272, for example to provide the functionality of the subject response input 214. Examples of user interface components 258 include display 274, keypad 276 and graphical user interface (GUI) 278, and may for example provide the functionality of the user interface 210. Embodiments of computer system 238 may include other conventional or otherwise known components to implement the methods in accordance with embodiments described herein.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled.

1. An audiometry method, comprising:
applying first test electrical stimulation to a subject, wherein the first test electrical stimulation is configured to stimulate hearing perception in the subject;
applying second test electrical stimulation to the subject, wherein the second test electrical stimulation is configured to stimulate hearing perception in the subject and is different than the first test electrical stimulation;
receiving from the subject an identification of one or more characteristics of the perceived hearing produced by the first test electrical stimulation and the second test electrical stimulation, wherein the one or more characteristics includes an origin location of the perceived hearing; and
recording the one or more characteristics of the perceived hearing identified by the subject in association with the first test electrical stimulation and the second test electrical stimulation.

2. The audiometry method of claim 1, wherein applying the first test electrical stimulation and the second test electrical stimulation includes simultaneously applying the first test electrical stimulation and the second test electrical stimulation.

3. The audiometry method of claim 1, wherein applying the first test electrical stimulation and the second test electrical stimulation includes sequentially applying the first test electrical stimulation and the second test electrical stimulation.

4. The audiometry method of claim 1, wherein applying the first test electrical stimulation and/or the second test electrical stimulation includes applying electrical stimulation corresponding to one or more of (1) a tone, (2) a range of tones, (3) speech, or (4) background noise.

5. The audiometry method of claim 1, wherein:
applying the first test electrical stimulation and/or the second test electrical stimulation includes applying the electrical stimulation corresponding to a range of intensities; and
receiving from the subject an identification of characteristics of the perceived hearing includes an identification of a threshold intensity at which the subject perceives the hearing.

6. The audiometry method of claim 1, wherein:
applying the first test electrical stimulation includes applying stimulation configured to at least predominately stimulate hearing on a first lateral side of the subject; and
applying the second test electrical stimulation includes applying stimulation configured to at least predominately stimulate hearing on a second lateral side of the subject that is different than the first lateral side.

7. The audiometry method of claim 6, wherein:
applying the first test electrical stimulation includes applying the first electrical stimulation to a first lateral side mastoid region of the subject; and
applying the second test electrical stimulation includes applying the second electrical stimulation to a second lateral side mastoid region of the subject.

8. The audiometry method of claim 1, wherein applying the first and second test electrical stimulation comprises applying electrical stimulation corresponding to a source of perceived hearing on a first lateral side of the subject. 9 The audiometry method of claim 1, wherein applying the first and second test electrical stimulation comprises applying electrical stimulation corresponding to a source of perceived hearing on a second lateral side of the subject.

10. The audiometry method of claim 1, wherein applying the first and second test electrical stimulation comprises applying electrical stimulation corresponding to a source of perceived hearing on an anterior side of the subject.

11. The audiometry method of claim 1, wherein applying the first and second test electrical stimulation comprises applying electrical stimulation corresponding to a source of perceived hearing on a posterior side of the subject.

12. The audiometry method of claim 1, wherein applying the first and second test electrical stimulation comprises applying electrical stimulation corresponding to a source of perceived hearing above the subject.

13. The audiometry method of claim 1, wherein applying the first and second test electrical stimulation comprises applying electrical stimulation corresponding to a source of perceived hearing below the subject.

14. The audiometry method of claim 1, wherein applying the first test electrical stimulation and the second test electrical stimulation comprises applying electrical stimulation corresponding to a stationary source of perceived hearing.

15. The audiometry method of claim 1, wherein applying the first test electrical stimulation and the second test electrical stimulation comprises applying electrical stimulation corresponding to a moving source of perceived hearing.

16. The audiometry method of claim 1, wherein the method further comprises:
applying at least a third test electrical stimulation to the subject, wherein the third test electrical stimulation is different than the first and second test electrical stimulation;
receiving from the subject an identification of characteristics of perceived hearing produced by the first, second and third test electrical stimulation; and
recording the characteristics of the perceived hearing identified by the subject in association with the first, second and third test electrical stimulation.

17. The audiometry method of claim 1, wherein applying the first and second test electrical stimulation comprises applying first and second stimulation having alterations in timing between the first and second stimulation.

18. The audiometry method of claim 1, wherein applying the first and second test electrical stimulation comprises applying first and/or second test electrical stimulation corresponding to increasing intensity with changing frequency.

19. The audiometry method of claim 1, further comprising:
applying one or more of:
test bone conduction stimulation to the subject, wherein the test bone conduction stimulation is configured to stimulate hearing perception in the subject; or
test air-conducted stimulation to the subject, wherein the test air-conducted stimulation is configured to stimulate hearing perception in the subject;
receiving from the subject an identification of characteristics of perceived hearing produced by the first and second test electrical stimulation and the one or more of the test bone conduction stimulation and the test air-conducted stimulation; and
recording the characteristics of the perceived hearing identified by the subject in association with the first and second test electrical stimulation and the one or more of the test bone conduction stimulation or the test air-conducted stimulation.

20. The audiometry method of claim 1, wherein applying the first and second test electrical stimulation includes:
applying the stimulation to at least predominately stimulate hearing on a first lateral side of the subject; and
applying the stimulation to mask the hearing on a second lateral side of the subject that is different that the first lateral side and that is produced by the stimulation on the first lateral side of the subject.

21. The audiometry method of claim 1, further comprising attenuating air conducted hearing of the subject while performing the method.

22. The audiometry method of claim 21, wherein attenuating air conducted hearing includes applying earplugs, ear muffs or other physical sound attenuating structures to one or more ears of the subject.

23. The audiometry method of claim 21, wherein attenuating air conducted hearing includes applying sound cancelling air conducted stimulation to one or more ears of the subject.

24. The audiometry method of claim 1, wherein the method is performed while the subject's ears are protected from ambient sound, for example by earmuffs, earplugs or noise cancellation.

25. The audiometry method of claim 1, for diagnosing tinnitus.

26. The audiometry method of claim 1, for diagnosing a site of lesion in the subject's ear organ, for example the outer ear, middle ear, or inner ear.
